(19) United States
(12) Patent Application Publication
(10) Pub. No.: US 2013/0065888 A1
(43) Pub. Date: Mar. 14, 2013

(54) OPHTHALMIC FORMULATIONS AND PROCESSES FOR THEIR PREPARATION

(71) Applicants: Biserka CETINA-CIZMEK, Zagreb (HR); Danka Bracko, Zagreb (HR); Sandra Miocic, Zagreb (HR); Iva Tunjic, Zagreb (HR)

(72) Inventors: Biserka CETINA-CIZMEK, Zagreb (HR); Danka Bracko, Zagreb (HR); Sandra Miocic, Zagreb (HR); Iva Tunjic, Zagreb (HR)

(21) Appl. No.: 13/585,379

(22) Filed: Aug. 14, 2012

Related U.S. Application Data
(60) Provisional application No. 61/588,444, filed on Jan. 19, 2012.
(60) Provisional application No. 61/523,467, filed on Aug. 15, 2011.

(51) Int. Cl. A61K 31/542; A61P 27/02

(52) U.S. Cl. 514/226.5

(57) ABSTRACT

The invention relates to a process for preparing ophthalmic formulations and to formulations containing a suspension of an ophthalmic drug in an aqueous vehicle. The invention further relates to the production of stable ophthalmic formulations that have a minimal propensity to form drug aggregates.

### CROSS-REFERENCE TO RELATED APPLICATIONS

This application claims the benefit of U.S. Provisional Application Nos. 61/588,444, filed Jan. 19, 2012; and 61/523,467, filed Aug. 15, 2011, each herein incorporated by reference in its entirety.

### FIELD OF THE INVENTION

The present invention relates to a process for preparing ophthalmic formulations and to formulations containing a suspension of an ophthalmic drug in an aqueous vehicle. In particular the invention relates to the production of stable ophthalmic formulations that have a minimal propensity to form drug aggregates.

### BACKGROUND OF THE INVENTION

Ophthalmic formulations wherein the ophthalmically active drug has a low water solubility, are typically manufactured as aqueous suspension formulations. These aqueous suspension formulations typically comprise a suspension of the drug or a mixture of drugs in an aqueous vehicle, wherein the aqueous vehicle contains dissolved excipients. The aqueous vehicle may also contain other water-soluble drugs. The drug particle sizes in the aqueous suspension need to be carefully controlled during manufacture. As well as ensuring the drug is stable with respect to chemical degradation, it is also important that the formulation is stable in the sense that drug particle aggregates do not form or are minimized either during the manufacture of the formulation, during storage of the formulation, and also during transport of the formulation, since it is known that shaking of such suspension formulations can cause drug particle aggregation.

Many drugs that are typically used in ophthalmic formulations, such as prostaglandins, carbonic anhydrase inhibitors, alpha-adrenergic agonists, non-steroidal anti-inflammatory agents, anti-fungal agents, antibiotics, corticosteroids and beta-blockers, particularly when in the form of their free bases or non-salt forms, have low water solubility at room temperature, and in some cases, are practically water insoluble at room temperature. Therefore such drugs need to be formulated as aqueous suspensions for topical use, e.g. eye drops.

Typical methods of manufacturing such ophthalmic suspension formulations include micronising the drug and adding this to an ophthalmically acceptable aqueous vehicle, or suspending the drug in the aqueous vehicle and milling the suspension using a ball mill. Sterilization of the formulation is usually achieved by heating (e.g. autoclaving) the mixture of the drug and aqueous vehicle before milling, or by heating the suspension of the drug after the milling step to produce a sterile final formulation. However, heating a final aqueous suspension for the purpose of providing a sterile formulation may cause partial or full dissolution of the drug in the aqueous vehicle and the resulting cooling may cause precipitation of drug particles in the form of crystals or aggregates in the sterilized product.

Alternatively, the constituents (e.g. the micronized drug and the aqueous vehicle) of the formulation can be separately sterilised and combined under aseptic conditions to produce a sterile final formulation. However, sterilisation of the micronized drug may cause chemical degradation or melting of the drug, and is therefore not suitable for all drugs.

EP2394637 discloses a process for sterilizing brinzolamide suspensions using gamma irradiation or ethylene oxide.

WO98/25620 proposes a method whereby suspension formulations containing brinzolamide, a carbonic anhydrase inhibitor, are made by process in which the first step involves autoclaving a concentrated slurry of brinzolamide in an aqueous suspension of specific surfactants, namely Tyloxapol or Triton X-100, in a milling bottle. The autoclaving temperature is typically above 120° C. The slurry is then subjected to a ball milling step at elevated temperature (i.e. above 80° C.), which reduces the particle size of the large brinzolamide crystals that form upon cooling of the hot slurry. The milled slurry is then passed though a screen having smaller openings than the milling bead size and added to the remaining constituents of the ophthalmic formulation under aseptic conditions. Finally, the milling beads are rinsed using sterile water and the mixture brought to final volume with water. WO98/25620 discloses that the use of other surfactants such as polysorbate 80, does not enable adequate particle size reduction of the brinzolamide crystals.

WO2006121963 discloses topical aqueous suspensions of sparingly soluble ophthalmic drugs such as nepafenac, containing a glycol and a poloxamer or meroxapol surfactant to enhance corneal penetration. The compositions are prepared by conventional methods wherein the drug particle size is typically reduced by, ball-milling using sizing beads, to a particle size range of from 0.1-100 μm, preferably 0.5-50 μm.

In these prior art procedures, the use of a typical ball milling process to reduce particle size of ophthalmic drugs in aqueous suspensions is not desirable for several reasons. Firstly, the ball-milling process and parameters must be carefully controlled in order to ensure adequate particle size reduction. As disclosed in WO 98/25620, this appears to be achievable only with a limited number of surfactants. Typically, the ball milling process is carried out at elevated temperature, particularly when the drug substance and wetting agent are previously sterilised by autoclaving. However, upon cooling to ambient temperature, agglomeration or crystallization of the suspended drug particles may still occur. This may necessitate conducting the ball milling process additionally during the cooling step, in order to minimize agglomeration or crystallisation. Further, ball milling requires the addition of milling beads, which themselves need to be sterilized to ensure that there is no introduction of foreign matter or contaminants into the milling composition. Additionally, due to the high friction forces present in the ball milling process, there is a risk of wearing of the milling balls and the resultant introduction of these as foreign matter into the product. This is obviously undesirable since small particles of foreign matter may act as nucleation sites and promote an undesirable nucleation or crystallisation of the drug product. Moreover, drug particles that have adhered to the beads during the ball milling process need to be recovered by rinsing. Due to the lack of water solubility of the drug, the rinsing may not be effective in removing all of the adhered drug particles, and hence it may be difficult to avoid the loss of some of the drug material.

As disclosed in WO98/25620, the ball-milling process does not sufficiently enable reduction of drug particle size when using a range of different excipients. For example, as discussed above, only certain specific surfactants can be used successfully in the ball milling process for preparing an ophthalmic suspension of brinzolamide, since other surfactants did not enable effective particle size reduction by the ball-mill. It was additionally found that the ball-milling process does not prevent subsequent aggregation of the drug particles in the suspension formulation. As a result, the suspension formulation may contain drug aggregates having a particle sizes above the recommended range for ophthalmic formulations. Thus, formulations prepared according to WO 98/25620 may not have the desired stability towards drug particle aggregation.

US20100297237 describes a pharmaceutical composition formed of nanoparticles, the nanoparticles comprising: (a) a poorly water soluble drug having a solubility in water of less than 5 mg/mL over the pH range of 6.5 to 7.5 at 25.degree. C., at least 90 wt % of the drug in the nanoparticles being non-crystalline; (b) a poorly water soluble non-ionizable polymer; and (c) an amine-functionalized methacrylate copolymer; wherein the nanoparticles have an average size of less than 500 nm; and the drug, the non-ionizable polymer, and the amine-functionalized methacrylate copolymer collectively constitute at least 80 wt % of the nanoparticles. According to US20100297237, it is apparently well known that the non-crystalline form of a low-solubility drug provides a greater aqueous concentration of drug relative to the crystalline form of the drug when administered to an aqueous use environment. Hence, US20100297237 describes the use of poorly aqueous soluble non-ionizable polymer in the nanoparticles stabilizes the poorly water soluble drug in the sense of reducing the rate of crystallization of the drug in the solid state and while in suspension in vivo.

US20100297237 discloses two processes, one is emulsification and the second is precipitation. Both require the poorly soluble drug to be in an organic solvent. The poorly water soluble non-ionizable polymer and amine-functionalized methacrylate copolymer are added to the organic solvent which is then mixed together with the aqueous vehicle to form a pre-emulsion which is them subjected to high pressure homogenisation to form a uniform emulsion. The use of organic solvents is preferably avoided in the pharmaceutical industry as is the inclusion of unnecessary excipients and processing steps.

Therefore there is a need to provide a more simple process for preparing ophthalmic suspensions which is more widely applicable. Moreover, there is a need to provide a process that can minimise or prevent the suspended drug particles from forming aggregates, e.g. upon storage and/or transportation.

### SUMMARY OF THE INVENTION

The present invention provides a process for preparing aqueous suspension formulations of an ophthalmic drug. In particular, ophthalmic formulations for topical application comprising: (i) an ophthalmic drug, (ii) at least one wetting agent, and (iii) an aqueous vehicle, wherein the ophthalmic drug is present as a suspension in the aqueous vehicle, can be manufactured by a process which comprises:
(a) subjecting a suspension of the ophthalmic drug in an aqueous solution of wetting agent to high pressure homogenization, and
(b) combining the mixture in step (a) with the aqueous vehicle.

The high pressure homogenization step enables the production of a stable suspension of the drug in the aqueous environment. Further, the high pressure homogenization step enables the drug particles in the suspension to have a more uniform particle size distribution compared to other methods such as ball milling and other high shear methods. Typically, the suspensions can be prepared far more quickly and efficiently than by ball milling. In the ball milling process disclosed in WO98/25620, the milling is carried out at elevated temperature (typically above 80° C.) for prolonged time periods (typically 18-19 hours) which may cause degradation of the drug, and carries a risk of the drug particles forming aggregates, particularly upon cooling of the milled solution. The present process is typically carried out at lower temperatures. Preferably the high pressure homogenization step in any embodiment of the present invention is carried out at a temperature of about 60° C. or less, preferably about 50° C. or less, more preferably about 40° C. or less, and most preferably about 30° C. or less, or about 28° C. or less. Preferably, the high pressure homogenization step is carried out in the absence of any applied heat source, i.e. around room temperature. Moreover, use of high pressure homogenization avoids the problems associated with ball milling such as the potential for introduction of contaminants and the possible loss of drug particles via the use of milling beads.

Furthermore, in contrast to the methods described in US20100297237 the present invention provides a favoured process which does not require the use of organic solvent and therefore releases from the concern of having organic residual solvent in an eye preparations, lowering potential toxic side effects and cellular damage at the ocular surface. The advantage of the present process is by preventing unnecessary risks to people and the environment. The present invention also avoids the need of preparing a starting organic solvent containing the drug and additional, unnecessary polymers.

A further advantage of using high pressure homogenization is that the extent of foaming of the suspension is lower particularly when compared to high shear homogenization. Hence, there is no need to employ an anti-foaming agent in the suspension. Additionally, the use of high pressure homogenization minimizes the heating of the suspension, and can minimise dissolution and subsequent crystallization of the drug, and also reduce the risk of thermal degradation of the drug.

The high pressure homogenization step employed in the present process can be used to reduce the particle size of the drug particles to a particle size range that is suitable for topical ophthalmic application, and/or to maintain the particle size of drug particles in the aqueous solution of wetting agent, wherein the drug particles have previously been micronized and thus already have a particle size range that is suitable for topical ophthalmic formulations. In particular, micronized drug particles, in their dry state, have a high propensity to agglomerate due to the high cohesive forces and high surface energy. Typically, micronized particles exist in a form of tightly bound agglomerates which are difficult to wet out and disperse into individual particles. Thus, for a previously micronized drug, the high pressure homogenization step described herein can effectively stabilize and prevent an already micronized drug from forming aggregates in an aqueous suspension without substantially reducing the particle size of the micronized drug particles. For example, in any embodiment of the present invention, the high pressure homogenization step may be carried on a suspension of the drug in the aqueous solution of wetting agent for the purpose of reducing or preventing the formation of drug particle aggregates without changing the starting particle size or particle size distribution of the drug in the aqueous solution of the wetting agent. This may be achieved, for example, by operating the homogenisation at a lower pressure and/or reduced number of cycles as described herein in order to achieve deagglomeration of the particles without effecting significant particle size reduction.

Preferably, in any embodiment of the present invention, the high pressure homogenization step is conducted at a suitable pressure and for a suitable number of cycles to effect deagglomeration of the drug particle agglomerates, without bringing about a significant particle size reduction of the individual particles.

In a high pressure homogenizer, product streams containing the suspended particles are collided at high pressure. The high pressure homogenization step can be carried out in any suitable high pressure homogenizer apparatus. Examples of these include microfluidizers and piston-gap homogenizers. Microfluidizers operate on a jet-stream principle, whereby the suspension is accelerated and forced into a homogenization chamber at high velocity and pressure. The chamber splits the suspension into two streams in order to reduce particle size and/or break up aggregates of previously-micronized particles. Piston-gap homogenizers involve maintaining the suspension in a cylinder of a larger diameter and forcing the suspension into a valve having a reduced diameter, which results in a large pressure and velocity increase. In any embodiment of the present invention as described herein microfluidization or piston-gap homogenization are the preferred methods for carrying out the high pressure homogenization. Microfluidization is particularly preferred.

The present invention further provides ophthalmic formulations wherein the ophthalmic drug is in aqueous suspension in the formulation and the formulation is substantially free of drug particles having a particle size of over 10 μm.

### BRIEF DESCRIPTION OF THE FIGURES

FIG. 1: Microscopic images of suspensions of brinzolamide with Polysorbate 80 as wetting agent, as prepared in Example 1.1 at 20× and 50× magnification

FIG. 2: Comparison of numerical particle size distribution of brinzolamide ophthalmic suspension samples prepared in Example 1.1

FIG. 3: Microscopic images of suspensions of brinzolamide with Poloxamer 407 as wetting agent, as prepared in Example 1.2 at 20× and 50× magnification

FIG. 4: Microscopic images of suspensions of brinzolamide with Tyloxapol as wetting agent, as prepared in Example 1.3 at 20× and 50× magnification

FIG. 5: Microscopic images of the suspension prepared in Example 2 taken: (a) at START point, (b) after shaking on a laboratory shaker (amplitude: 90 min⁻¹) for 24 h, and (b) after shaking on a laboratory shaker (amplitude: 90 min⁻¹) for 48 h.

### DETAILED DESCRIPTION OF THE INVENTION

As used herein, unless indicated otherwise, “room temperature” refers to typical ambient temperatures, i.e. temperatures in the range of about 18 to about 30° C., preferably about 20 to about 25° C., and more preferably about 25° C.

As used herein, unless indicated otherwise, percentages refer to weight % based on weight of the total formulation.

Preferably, particle size analyses and particle size distributions of the suspension are obtained by microscopy using a particle size analyser (preferably by following US Pharmacopeia 29 <776>—Optical Microscopy).

The ophthalmic drug in the formulation is present as a suspension in the aqueous vehicle. Hence the ophthalmic drugs in the processes and formulations of the present invention have a low water-solubility, or are sparingly soluble in water, or are practically insoluble in water at room temperature. Typically, such drugs have a water-solubility at 25° C. of about 0.001 to about 1% (w/v), about 0.001 to about 0.5% (w/v), about 0.001 to about 0.2% (w/v), about 0.001 to about 0.1% w/v or about 0.001 to about 0.05% w/v.

As used herein, the term “drug particles” is intended to refer to all suspended drug particles including agglomerated drug particles.

The process of the present invention enables the production of an aqueous suspension formulation of an ophthalmic drug for topical application, wherein the formulation comprises: (i) an ophthalmic drug, (ii) at least one wetting agent, and (iii) an aqueous vehicle, wherein the ophthalmic drug is present as a suspension in the aqueous vehicle. The process comprises:

(a) subjecting a suspension of the ophthalmic drug in an aqueous solution of wetting agent to high pressure homogenization, and

(b) combining the mixture in step (a) with the aqueous vehicle.

The high pressure homogenization may be carried out by any high pressure homogenization apparatus, including a microfluidizer or a piston-gap homogenizer. Microfluidization is the preferred high pressure homogenization method for the process of the present invention.

In any embodiment of the present invention, the high pressure homogenization step may be carried out at a temperature of about 60° C. or less, preferably about 50° C. or less, more preferably about 40° C. or less, and most preferably about 30° C. or less, or about 28° C. or less. Preferably, the high pressure homogenization step is carried out in the absence of any applied heat source, i.e. at ambient temperature/room temperature.

In any embodiment of the present invention, the high pressure homogenization is preferably conducted at a pressure of at least about 100 bar, at least about 150 bar, at least about 200 bar, at least about 250 bar, at least about 300 bar, at least about 400 bar, preferably at least about 500 bar, at least about 600 bar, at least about 700 bar, at least about 800 bar or at least about 1000 bar. For example, the high pressure homogenization is conducted at a pressure of about 100 to about 3100 bar, about 100 to about 3000 bar, about 100 to about 2000 bar, about 100 to about 1500 bar, about 200 to about 1500 bar, about 200 to about 1000 bar, about 200 to about 750 bar, about 300 to about 1500 bar, about 300 to about 1000 bar, about 300 to about 750 bar, about 300 to about 600 bar, about 300 to about 550 bar, about 300 to about 500 bar, about 400 to about 1000 bar, about 400 to about 600 bar, about 500 to about 1000, or about 500 to about 800 bar. More preferably, in any embodiment of the process of the present invention, the high pressure homogenization is conducted at a pressure of about 100 to about 500 bar, particularly about 150 to about 450 bar, more particularly about 200 to about 400 bar, and preferably about 300 to about 380 bar.

Preferably, in any embodiment of the present invention, the high pressure homogenization is carried out for at least about 2, at least about 3, at least about 5, at least about 6, at least about 7, at least about 8, at least about 10, at least about 12, or at least about 20 cycles.

For example, in any embodiment of the present invention, the high pressure homogenization may be carried out over about 2 to about 15 cycles, about 2 to about 10 cycles, about 2 to about 6 cycles, about 3 to about 10 cycles, about 4 to about 8 cycles, or about 4 to about 10 cycles.

In a typical process of any embodiment of the present invention, the high pressure homogenization is conducted at pressure of about 200 to about 400 bar, for about 3 to about 7 cycles. Alternatively, in any embodiment of the present invention, the high pressure homogenization is conducted at a pressure of about 300 to about 380 bar for about 4 to about 6 cycles.

Preferably, in any embodiment of the present invention, the suspension of the ophthalmic drug in the aqueous solution of wetting agent is prepared by adding the solid drug to the aqueous solution of the wetting agent. Preferably, the suspension of the ophthalmic drug in the wetting agent solution is substantially free of organic solvent and/or water insoluble polymers (or poorly aqueous soluble polymers).

Preferably, the suspension of the ophthalmic drug in the aqueous solution of wetting agent is sterilized prior to high pressure homogenization. The sterilization may be conducted by dry-sterilization of the drug particles, and sterilization of the aqueous solution of wetting agent, and combining the sterilized components under aseptic conditions before micronization or homogenization. Autoclaving is a preferred method of sterilization of the aqueous solution of the wetting agent in the processes of the present invention.

The autoclaving may be conducted at sufficient temperatures and time periods in order to obtain a sterile material. Typically, exposure to the minimum temperature (e.g. about 100 to about 150° C., preferably about 110 to about 140° C., more preferably about 120 to about 130° C.) and minimum time that is effective to obtain a sterile material is preferred. Typically, the autoclaving is conducted for about 10 to about 30 minutes, more preferably about 10 to about 20 minutes. For example, the autoclaving can be conducted at a temperature of about 115° C. to about 125° C. for about 10 to 20 minutes (e.g. about 118° C. to about 125° C. for about 15 minutes.

The dry-sterilization of the drug particles may be conducted by any suitable sterilization process appropriate for the drug. These can include, for example, dry heat at a suitable temperature and for a suitable period of time, gamma radiation, electron beam radiation, gamma radiation, sterile filtration, and treatment with ethylene oxide. Treatment with ethylene oxide has been found to be a particularly preferred sterilization method for brinzolamide.

The aqueous vehicle component of the formulation can be prepared by a process comprising: (i) forming an aqueous slurry containing an ophthalmically acceptable excipient selected from the group consisting of a chelating agent, preservative, tonicity agent, viscosity/suspending agent, and optionally a buffer, or a mixture thereof, (ii) adjusting the aqueous slurry to an ophthalmically acceptable pH, and (iii) sterilizing the slurry. The aqueous vehicle component can be conveniently sterilized by autoclaving as described above.

The combining of the homogenized suspension and the aqueous vehicle can then be carried out under known aseptic techniques. For example, steps (a) and/or (b) are carried out under aseptic conditions.

Alternatively, it may be convenient to first obtain a suspension of the drug particles in the aqueous solution of wetting agent, and subjecting the suspension to sterilization. The suspension can be sterilized by any suitable method as described above. In some instances, particularly where autoclaving is used, the particle size of the drug may be increased. In this case, the suspension can then be conveniently treated to high pressure homogenization. The high pressure homogenization may be operated at higher pressure and/or increased number of cycles in order to achieve the desired micronized particles. For example, the high pressure homogenization may be conducted at pressures of about 500 to about 3200 bar, preferably about 1000 to about 3000 bar, more preferably about 1500 to about 2500 bar. Using any of these pressure ranges, the number of cycles can be from about 3 to about 12, from about 5 to about 10, and preferably from about 6 to about 8.

In a preferred embodiment of the present invention, the drug is dry-sterilized as described above, the solution of the wetting agent is sterilized as described above, and the sterilized components are mixed under aseptic conditions before subjecting the mixture to high pressure homogenization. The high pressure homogenization can be conducted under the conditions described herein. Preferably, the high pressure homogenization is conducted at pressure of about 100 to about 500 bar for about 3 to about 8 cycles, or about 200 to about 400 bar, for about 3 to about 7 cycles. Alternatively, in any embodiment of the present invention, the high pressure homogenization is conducted at a pressure of about 300 to about 380 bar for about 4 to about 6 cycles.

The process of the present invention is applicable to the production of any ophthalmic drug that is formulated as an aqueous suspension, i.e. drugs having a low aqueous solubility or drugs that are practically insoluble in water. The ophthalmic drug is preferably selected from the group consisting of a prostaglandin, carbonic anhydrase inhibitor, α-adrenergic agonist, non-steroidal anti-inflammatory, anti-fungal agent, antibiotic, corticosteroid, beta-blocker, or a combination thereof. In a preferred embodiment, the ophthalmic drug is micronized prior to addition to the wetting agent.

Examples of such ophthalmic drugs include those selected from the group consisting of brimonidine, brinzolamide, dorzolamide, natamycin, ofloxacin, bimatoprost, travoprost, latanoprost, nepafenac, ketoconazole, fluconazole, voriconazole, hydrocortisone, prednisolone, dexamethasone, timolol, levobunolol, betaxolol, or pharmaceutically acceptable salts thereof.

Preferred classes of ophthalmic drugs suitable for the present process include those selected from the group consisting of a prostaglandin, carbonic anhydrase inhibitor, α-adrenergic agonist, antibiotic or beta-blocker, or a combination thereof, and more preferably a prostaglandin, carbonic anhydrase inhibitor, α-adrenergic agonist or antibiotic, optionally in combination with a beta-blocker.

In particularly preferred embodiments of the present invention, the ophthalmic drug is bimatoprost, brimonidine, brinzolamide, dorzolamide, latanoprost, ofloxacin, and travoprost or a pharmaceutically acceptable salt thereof, optionally in combination with a beta-blocker. Preferred beta-blockers for use in the combination formulations are those selected from the group consisting of levobunolol, timolol or betaxolol, or a pharmaceutically acceptable salt thereof. Timolol, or its pharmaceutically acceptable salt thereof, preferably timolol maleate. Preferred α-adrenergic agonists include brimonidine or a pharmaceutically acceptable salt thereof, preferably brimonidine tartrate.

In any embodiment of the present invention, the preferred ophthalmic agents are carbonic anhydrase inhibitor. More preferably the ophthalmic agent is selected from the group consisting of brinzolamide and dorzolamide, or pharmaceutically acceptable salts thereof, optionally in combination with timolol or a pharmaceutically acceptable salt thereof (preferably timolol maleate), or in combination with brimonidine or a pharmaceutically acceptable salt thereof (preferably brimonidine tartrate). Particularly preferred combination formulations of the present invention are brinzolamide and timolol (preferably timolol maleate) or brinzolamide and brimonidine (preferably brimonidine tartrate).

An especially preferred ophthalmic drug is brinzolamide, or a pharmaceutically acceptable salt thereof, preferably in combination with timolol maleate.

Brinzolamide is preferably present in an amount of about 10 mg/ml of the pharmaceutical formulation.

In the processes and formulations of the present invention, it is preferred that the ophthalmic drug (e.g. brinzolamide, dorzolamide, etc) is suspended in the aqueous vehicle. The additional drug, when the formulation is a combination containing at least one other drug, can either be in suspension, or preferably, is dissolved in the aqueous vehicle. For example, in a preferred embodiment, where the ophthalmic drug is brinzolamide in combination with timolol maleate, the brinzolamide (being poorly water-soluble) is present as a suspension, whereas the timolol maleate (being water-soluble) is dissolved in the aqueous vehicle. Preferably in this combination, the ophthalmic formulation contains about 10 mg/ml of brinzolamide and about 5 mg/ml of timolol (as the maleate salt). In another preferred embodiment, wherein the ophthalmic drug is brinzolamide in combination with brimonidine tartrate, the brinzolamide is present as a suspension, and the brimonidine tartrate is dissolved in the aqueous vehicle. Preferably in this combination, the ophthalmic formulation contains about 10 mg/ml of brinzolamide and about 2 mg/ml of brimonidine (as the tartrate salt).

In any embodiment of the present invention the wetting agent is preferably a non-ionic wetting agent. Preferably, the wetting agent is water soluble or swellable. More preferably the wetting agent is water soluble. “Water soluble” is to be understood in the manner used in standard texts such as the “Handbook of Pharmaceutical Excipients” (Raymond C Rowe, Paul J Sheskey and Siân C Owen, Fifth Edition, Pharmaceutical Press and American Pharmacists Association 2006)

Suitable classes of wetting agents include those selected from the group consisting of polyoxypropylene-polyoxyethylene block copolymers (poloxamers), polyethoxylated ethers of castor oils, polyoxyethylenated sorbitan esters (polysorbates), polymers of oxyethylated octyl phenol (Tyloxapol), polyoxyl 40 stearate, fatty acid glycol esters, fatty acid glyceryl esters, sucrose fatty esters, polyoxyethylene fatty esters, and mixtures thereof.

Specific examples of suitable wetting agents include those selected from the group consisting of: polyoxyethylene-polyoxypropylene block copolymers (poloxamers) such as: polyoxyethylene (160) polyoxypropylene (30) glycol [Pluronic F68], polyoxyethylene (42) polyoxypropylene (67) glycol [Pluronic P123], polyoxyethylene (54) polyoxypropylene (39) glycol [Pluronic P85], polyoxyethylene (196) polyoxypropylene (67) glycol [Poloxamer 407, Pluronic F127] and polyoxyethylene (20) polyoxypropylene (20) glycol [Pluronic L44], polyoxyethylenated sorbitan esters (polysorbates) such as poly(oxyethylene)sorbitan monopalmitate (polysorbate 40), poly(oxyethylene)sorbitan monostearate (polysorbate 60), poly(oxyethylene)sorbitan tristearate (polysorbate 65), poly(oxyethylene)sorbitan monooleate (polysorbate 80), poly(oxyethylene)sorbitan monolaurate, and poly(oxyethylene)sorbitan trioleate, polyethoxylated ethers of castor oils such as polyoxyethylene hydrogenated castor oil 10, polyoxyethylene hydrogenated castor oil 40, polyoxyethylene hydrogenated castor oil 50 and polyoxyethylene hydrogenated castor oil 60, polyoxyl 40 stearate, sucrose fatty esters and polyoxyethylene fatty esters and mixtures thereof.

Preferably, the wetting agent is selected from the group consisting of: polyoxyethylene-polyoxypropylene block copolymers (poloxamers) such as: polyoxyethylene (160) polyoxypropylene (30) glycol [Pluronic F68], polyoxyethylene (42) polyoxypropylene (67) glycol [Pluronic P123], polyoxyethylene (54) polyoxypropylene (39) glycol [Pluronic P85], polyoxyethylene (196) polyoxypropylene (67) glycol [Poloxamer 407, Pluronic F127] and polyoxyethylene (20) polyoxypropylene (20) glycol [Pluronic L44], polyoxyethylenated sorbitan esters (polysorbates) such as poly(oxyethylene)sorbitan monopalmitate (polysorbate 40), poly(oxyethylene)sorbitan monostearate (polysorbate 60), poly(oxyethylene)sorbitan tristearate (polysorbate 65), poly(oxyethylene)sorbitan monooleate (polysorbate 80), poly(oxyethylene)sorbitan monolaurate, and poly(oxyethylene)sorbitan trioleate and mixtures thereof.

More preferably, the wetting agent is a polyoxyethylene-polyoxypropylene block copolymer (poloxamer). Examples of suitable poloxamers include: polyoxyethylene (160) polyoxypropylene (30) glycol [Pluronic F68], polyoxyethylene (42) polyoxypropylene (67) glycol [Pluronic P123], polyoxyethylene (54) polyoxypropylene (39) glycol [Pluronic P85], polyoxyethylene (196) polyoxypropylene (67) glycol [Poloxamer 407, Pluronic F 127] and polyoxyethylene (20) polyoxypropylene (20) glycol [Pluronic L44] or a mixture thereof.

Further preferred are wetting agents selected from the group consisting of polyoxyethylene (42) polyoxypropylene (67) glycol [Pluronic P123], polyoxyethylene (54) polyoxypropylene (39) glycol [Pluronic P85], polyoxyethylene (196) polyoxypropylene (67) glycol [Poloxamer 407, Pluronic F127] and mixtures thereof.

An especially preferred wetting agent is polyoxyethylene (196) polyoxypropylene (67) glycol [Poloxamer 407, Pluronic F127].

The aqueous vehicle component of the ophthalmic formulation preferably comprises water and at least one ophthalmically acceptable excipient. Preferably, the aqueous vehicle comprises a solution of the one or more ophthalmically acceptable excipients in water.

Suitable ophthalmically acceptable excipients include those selected from the group consisting of a chelating agent, preservative, tonicity agent, viscosity/suspending agent, buffer, pH modifying agent, or a mixture thereof.

Preferably, the ophthalmically acceptable excipient is selected from the group consisting of a chelating agent, preservative, tonicity agent, viscosity/suspending agent and pH modifying agent, or a mixture thereof.

As to chelating agents, any suitable ophthalmically acceptable chelating agent can be used. Examples of these include those selected from the group consisting of ethylenediaminetetraacetic acid and metal salts thereof, such as disodium edetate, trisodium edetate, tetrasodium edetate or mixtures thereof. Disodium edetate is a particularly preferred chelating agent.

The chelating agent(s) may be added in an amount of about 0.005 to about 0.05 wt %, preferably about 0.005 to about 0.02 wt %, and more preferably about 0.008 to about 0.015 wt %.

Preferably, the aqueous vehicle includes a preservative. Preferred preservatives include those selected from the group consisting of quaternary ammonium salts such as benzalkonium halides (preferably benzalkonium chloride), chlorhexidine gluconate, benzethonium chloride, cetyl pyridinium chloride, benzyl bromide, phenylmercury nitrate, phenylmercury acetate, thiomerosal, merthiolate, phenylmercuryborate, methylparaben, propylparaben, sorbic acid, potassium sorbate, sodium benzoate, sodium propionate, ethyl p-hydroxybenzoate, butyl-p-hydroxybenzoate, sorbic acid, or mixtures thereof. More preferably, the preservative is a quaternary ammonium salt such as benzalkonium halides (preferably benzalkonium chloride), chlorhexidine gluconate, benzethonium chloride, cetyl pyridinium chloride, potassium sorbate, sodium benzoate, ethyl p-hydroxybenzoate, butyl p-hydroxybenzoate, or mixtures thereof. Benzalkonium chloride is an especially preferred preservative.

The preservative(s) may be used in an amount of about 0.005 to about 0.05 wt %, preferably about 0.005 to about 0.02 wt %, and more preferably about 0.008 to about 0.015 wt %.

The aqueous vehicle may also include a tonicity agent to adjust the tonicity (osmotic pressure) in order to achieve an ophthalmically compatible formulation. Preferably, the tonicity agent is selected from a glycol (such as propylene glycol, diethylene glycol, triethylene glycol), glycerol, dextrose, glycerin, mannitol, potassium chloride and sodium chloride or a mixture thereof. Preferably the tonicity agent is selected from the group consisting of glycerin, mannitol, potassium chloride, and sodium chloride. More preferably mannitol and/or sodium chloride (and most preferably a mixture thereof) are employed.

The tonicity agent(s) is preferably used in an amount of about 0.5 to about 8 wt %, preferably about 1 to about 6 wt %, and more preferably about 2 to about 4 wt %.

When a mixture of mannitol and sodium chloride is used as tonicity agents, preferably the weight ratio of mannitol:sodium chloride is about 4:1 to about 15:1, more preferably about 6:1 to about 14:1, or 8:1 to about 14:1 and particularly about 10:1 to about 12:1.

If mannitol alone is used as the tonicity agent, it is preferably used in an concentration of about 4.5 to about 6.5 wt %, and more preferably in a concentration of about 5.0 to about 5.5 wt %. If sodium chloride alone is used as the tonicity agent, it is preferably used in a concentration of about 0.7 to about 1.0 wt %, and more preferably in a concentration of about 0.8 to about 0.9 wt %.

The aqueous vehicle preferably also contains a viscosity/suspending agent. Suitable viscosity/suspending agents include those selected from the group consisting of cellulose derivatives, such as methyl cellulose, ethyl cellulose, hydroxyethylcellulose, polyethylene glycols (such as polyethylene glycol 300, polyethylene glycol 400), carboxymethyl cellulose, hydroxypropylmethyl cellulose, cross-linked acrylic acid polymers (carbomers), such as polymers of acrylic acid cross-linked with polyalkenyl ethers or divinyl glycol (Carbopols—such as Carbopol 934, Carbopol 934P, Carbopol 971, Carbopol 974 and Carbopol 974P) or a mixture thereof. In preferred embodiments of the present invention, the viscosity/suspending agent is a carbomer, preferably Carbopol 974P.

The viscosity/suspending agent(s) may be present in an amount of about 0.05 to about 2 wt %, preferably 0.1 to about 1 wt %, more preferably about 0.2 to about 0.8 wt % and most preferably about 0.3 to about 0.5 wt %.

In order to adjust the formulation to an ophthalmically acceptable pH (typically a pH range of about 5.0 to about 9.0, more preferably about 5.5 to about 8.5, particularly about 6.0 to about 8.5, about 7.0 to about 8.5, about 7.2 to about 7.7, about 7.1 to about 7.9, or about 7.5 to about 8.0) the formulation may contain a pH modifying agent.

The pH modifying agent is typically a mineral acid or metal hydroxide base preferably selected from the group of potassium hydroxide, sodium hydroxide, hydrochloric acid, or mixtures thereof, and preferably sodium hydroxide and/or hydrochloric acid. These acidic and/or basic pH modifying agents are added to adjust the formulation to the target ophthalmically acceptable pH range. Hence it may not be necessary to use both acid and base—depending on the formulation, the addition of one of the acid or base may be sufficient to bring the mixture to the desired pH range.

The aqueous vehicle may also contain a buffer to stabilize the pH. When used, the buffer is preferably selected from the group consisting of a phosphate buffer (such as sodium dihydrogen phosphate and disodium hydrogen phosphate), a borate buffer (such as boric acid, or salts thereof including disodium tetraborate), citrate buffers (such as citric acid, or salts thereof including sodium citrate) and ε-aminocaproic acid, or mixtures thereof.

In particularly preferred embodiments of the present invention the aqueous vehicle comprises water, benzalkonium chloride, disodium edetate, sodium chloride, mannitol, carbomer (preferably Carbopol 974P). Optionally sodium hydroxide and/or hydrochloric acid are added in order to achieve an ophthalmically acceptable pH range. Typically, the pH is adjusted to achieve an ophthalmically acceptable pH range using either sodium hydroxide and/or hydrochloric acid.

Advantageously, the process enables the production of an ophthalmic suspension formulation wherein the ophthalmic drug is substantially free of drug particles having a particle size of over 10 μm. For example, by “substantially free of drug particles having a particle size of over 10 μm”, it is meant that as observed by microscopy particle size analysis, the formulation contains less than 0.02%, less than 0.01%, less than 0.005%, less than 0.002%, and preferably less than 0.0001% by weight of drug particles having a particle size of over 10 μm. More preferably, the ophthalmic suspension formulation contains no detectable drug particles having a particle size of 10 μm or more (e.g. as observed by microscopy particle size analysis, there are no detectable drug particles having a particle size of over 10 μm).

In any embodiment of the present invention the particle size ranges of the ophthalmic drug in the formulation are: less than about 10 μm, preferably about 9.5 μm or less, about 8.5 μm or less, or about 8.0 μm or less. Typical average particle size ranges are about 0.1 to less than about 10 μm, about 0.1 to about 9.5 μm, about 0.2 to about 9.0 μm, about 0.5 to about 9.0 μm, about 0.8 to about 8.5 μm, about 0.9 to about 8.0 μm, and particularly about 1 to about 7.5 μm.

The applicant has found that the use of other techniques such as ball milling or high shear homogenization does not enable the production of suspensions having uniform particle size distributions. In particular, suspensions prepared by ball milling or high shear homogenization can contain a percentage of particles (e.g. drug aggregates) having particle sizes of 10 μm or more. Such particle sizes, even when present in low concentrations, are undesirable in ophthalmic formulations, since these can cause undesirable side effects such as discomfort at the site of application, or eye irritation.

Moreover, as discussed above, the use of high pressure homogenization in accordance with the process of the present invention enables the production of a stable suspension formulation. In particular, the formulations are stable in the sense that they have a significantly lower propensity to form aggregates in contrast to formulations prepared by other procedures such as ball milling and high shear homogenization.

The suspension stability of formulations prepared by the process of the present invention can be determined by shaking the formulation at an amplitude of 90/min for 24 hours at room temperature, preferably at an amplitude of 90/min for 48 hours at room temperature. Formulations of the present invention have been found to be substantially free (for example, have less than 0.02%, less than 0.01%, less than 0.005%, less than 0.002%, or less than 0.0001% by weight of drug particles having a particle size of 10 μm or more, and preferably having no detectable drug particles having a particle size of 10 μm or more) following shaking the formulation at an amplitude of 90/min for 24 hours at room temperature, preferably following shaking the formulation at an amplitude of 90/min for 48 hours at room temperature.

The process of the present invention is particularly suitable for the preparation of multiple dose or single dose eye drops, and preferably a multiple dose eye drop.

The present invention further encompasses ophthalmic formulations obtainable by a process as defined in any of the embodiments described herein.

A further aspect of the present invention is the provision of an ophthalmic formulation for topical application comprising: (i) an ophthalmic drug, (ii) at least one wetting agent, and (iii) an aqueous vehicle, wherein the ophthalmic drug is in aqueous suspension in the formulation, and wherein the formulation is substantially free of drug particles having a particle size of over 10 μm.

Preferably, the formulation contains less than 0.02%, less than 0.01%, less than 0.005%, less than 0.002%, or less than 0.0001% by weight of drug particles having a particle size of over 10 μm. More preferably, the formulation contains no detectable drug particles having a particle size of over 10 μm.

Preferably, the formulation is substantially free of organic solvent and/or poorly aqueous soluble polymers. By “poorly aqueous soluble” is meant that the polymer has a solubility of less than 0.1 mg/mL when administered alone at a concentration of 0.2 mg/mL to a phosphate buffered saline solution (PBS) at pH 6.5. An appropriate PBS solution is an aqueous solution comprising 20 mM sodium phosphate (Na.sub.2HPO.sub.4), 47 mM potassium phosphate (KH.sub.2PO.sub.4), 87 mM NaCl, and 0.2 mM KCl, adjusted to pH 6.5 with NaOH.

In particularly preferred embodiments, the formulation according to any of the above embodiments is substantially free of drug particles having a particle size of 10 μm or more following shaking the formulation (preferably 15 ml of the formulation in a 30 ml glass bottle) at an amplitude of 90/min for 24 hours at room temperature, preferably following shaking the formulation at an amplitude of 90/min for 48 hours at room temperature.

In further preferred embodiments, the formulation according to any of the above embodiments have less than 0.02%, less than 0.01%, less than 0.005%, less than 0.002%, or less than 0.0001% by weight of drug particles having a particle size of 10 gm or more following shaking the formulation (preferably 15 ml of the formulation in a 30 ml glass bottle) at an amplitude of 90/min for 24 hours at room temperature, preferably following shaking the formulation at an amplitude of 90/min for 48 hours at room temperature.

In a particularly preferred embodiment of the formulation according to any of the above embodiments, the formulation has no detectable drug particles having a particle size of 10 μm or more following shaking the formulation (preferably 15 ml of the formulation in a 30 ml glass bottle) at an amplitude of 90/min for 24 hours at room temperature, preferably following shaking the formulation at an amplitude of 90/min for 48 hours at room temperature.

The formulation according to any aspect of the present invention is preferably a multiple dose or single dose eye drop, preferably a multiple dose eye drop.

In any embodiment of the formulation according to the present invention, the ophthalmic drug is as defined as set out in reference to any of the embodiments as described herein for the process. Preferably, the ophthalmic drug is brinzolamide or dorzolamide, or pharmaceutically acceptable salts thereof, optionally in combination with timolol (preferably timolol maleate) or brimonidine (preferably brimonidine tartrate). More preferably, the ophthalmic drug is brinzolamide, brinzolamide in combination with timolol maleate, or brinzolamide in combination with brimonidine tartrate.

In any embodiment of the formulation of the present invention, the wetting agent and aqueous vehicle components of the formulation are as defined as set out herein in relation to the process of the present invention.

The present invention also encompasses the use of high pressure homogenization to prevent formation of drug aggregates in an ophthalmic formulation containing an ophthalmic drug suspended in an aqueous vehicle containing at least one wetting agent. In particular, the high pressure homogenization step may be used to prevent drug aggregates forming in the ophthalmic formulation when the drug particles are already present in micronized form, i.e. having particles sizes suitable for topical ophthalmic application.

Thus, in any embodiment of the process and use described herein, the high pressure homogenization is applied to the suspension containing the premicronized drug in the aqueous solution of wetting agent, and does not bring about particle size reduction, but instead, stabilises the already micronized drug, and thus prevents the formation of drug aggregates. Additionally, the high pressure homogenization ensures effective coating of the drug particles with the wetting agents/surfactants, which has been found to advantageously stabilise the suspension and prevent formation of drug aggregates. The stability of the micronized drug suspension can be determined by the tests described above, i.e. by shaking the formulation (preferably 15 ml of the formulation in a 30 ml glass bottle) at an amplitude of 90/min for 24 hours at room temperature, preferably following shaking the formulation (preferably 15 ml of the formulation in a 30 ml glass bottle) at an amplitude of 90/min for 48 hours at room temperature.

Preferably, the use of high pressure homogenization produces a formulation which is substantially free of drug particles having a particle size of 10 μm or more following shaking the formulation (preferably 15 ml of the formulation in a 30 ml glass bottle) at an amplitude of 90/min for 24 hours at room temperature, preferably following shaking the formulation (preferably 15 ml of the formulation in a 30 ml glass bottle) at an amplitude of 90/min for 48 hours at room temperature. More preferably, the formulation contains less than 0.02%, less than 0.01%, less than 0.005%, less than 0.002%, or less than 0.0001% by weight of drug particles having a particle size of 10 μm or more following shaking the formulation at an amplitude of 90/min for 24 hours at room temperature, preferably following shaking the formulation (preferably 15 ml of the formulation in a 30 ml glass bottle) at an amplitude of 90/min for 48 hours at room temperature. More preferably, following the high pressure homogenization, the formulation has no detectable drug particles having a particle size of 10 μm or more following shaking the formulation (preferably 15 ml of the formulation in a 30 ml glass bottle) at an amplitude of 90/min for 24 hours at room temperature, preferably following shaking the formulation (preferably 15 ml of the formulation in a 30 ml glass bottle) at an amplitude of 90/min for 48 hours at room temperature.

Preferably, the drug is as defined in any of the above embodiments as set herein in respect of the process or formulation. More preferably, the drug is a carbonic anhydrase inhibitor selected from brinzolamide or dorzolamide (more preferably brinzolamide), or pharmaceutically acceptable salts thereof. Brinzolamide is a particularly preferred drug according to any embodiment of the present invention as described herein.

For the avoidance of doubt, additional embodiments of the present invention include those where each use of the term “comprising” is replaced with “consisting of” or “consisting essentially of” with such terms having their generally accepted meanings.

The present invention is illustrated by the following examples, which are not intended to limit the scope of the invention. It will be appreciated that various modifications are within the spirit and scope of the invention.

### EXAMPLES

### Example 1

### Effect of Different Homogenization Techniques on Particle Size Distribution of Brinzolamide in Brinzolamide Ophthalmic Suspension

In Examples 1.1, 1.2 and 1.3, brinzolamide ophthalmic suspensions were prepared by using three different homogenization techniques (high pressure homogenization in accordance with the present invention, high shear homogenization and ball milling) in order to test the effectiveness these techniques on de-agglomeration of brinzolamide particles and coating of the particles with wetting agent. Three different wetting agents were used (Polysorbate 80, Poloxamer 407 and Tyloxapol). Particle size analyses were conducted by microscopy.

### Example 1.1

### Wetting Agent—Polysorbate 80

In this example, a brinzolamide ophthalmic suspension was prepared in a 0.05% aqueous solution of Polysorbate 80 as wetting agent by the following three methods Prior to the treatment below, the suspension contained agglomerates of brinzolamide wherein the agglomerates have a particle size of greater than 10 μm (and hence are unsuitable for use in topical ophthalmic formulations), wherein the particle size distribution of the individual brinzolamide particles is d₉₀≦3.0 μm. Portions of the suspension were treated separately by the following three methods (i)-(iii):
(i) high pressure homogenization (5000 psi/5 cycles using Microfluidizer® high pressure homogenizer)
(ii) high shear homogenization (8000 rpm/120 minutes (Ultra-Turrax high shear mixer)
(iii) ball milling (8.5 hours at 22-24 rpm (tumble blender; Zirconox beads: 0.7-1.2 mm)

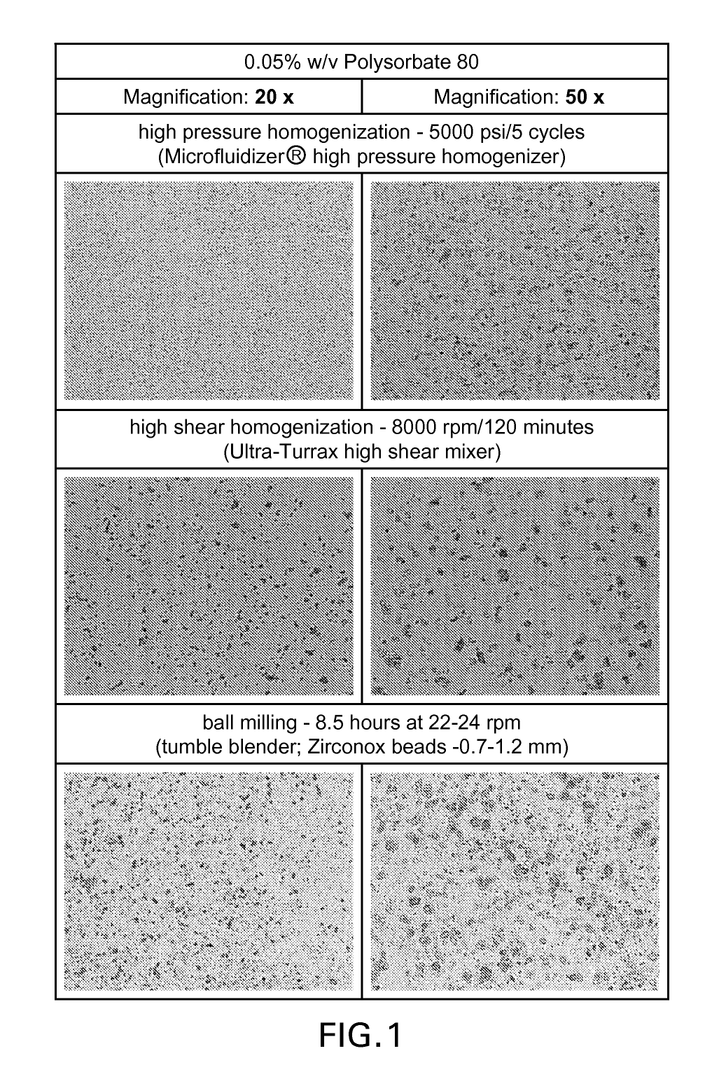

Microscopic images of the suspensions at 20× and 50× magnification are shown in FIG. 1.

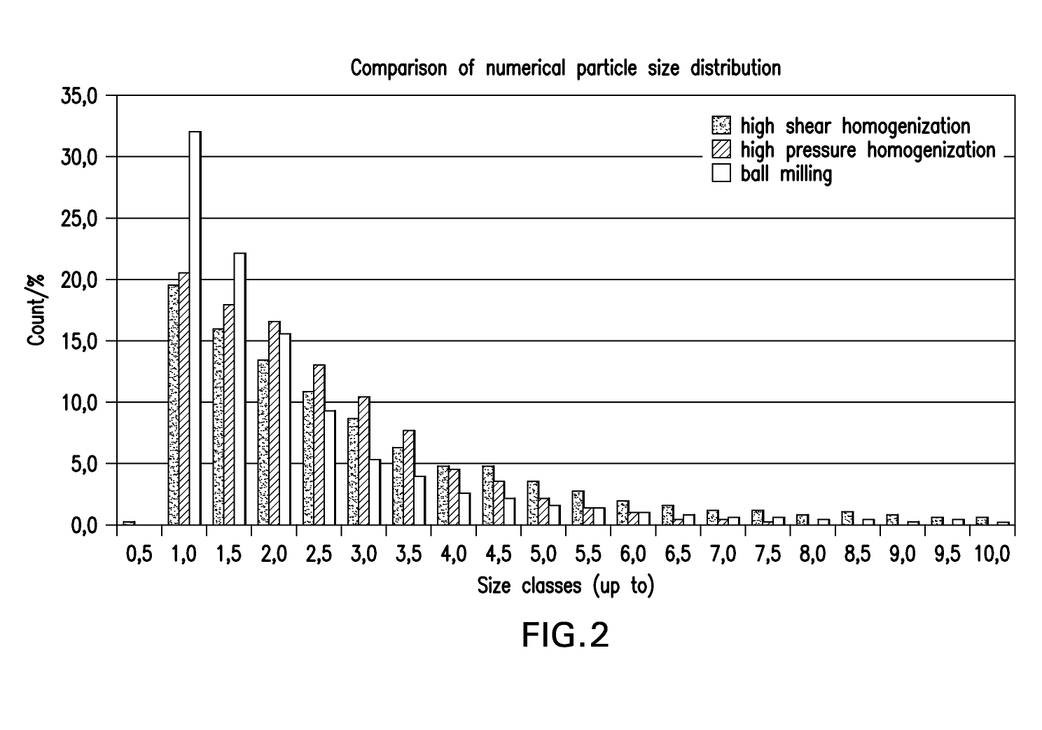

A comparison of the numerical particle size distribution of brinzolamide ophthalmic suspension samples prepared by high pressure homogenization, high shear homogenization and ball milling is presented graphically in FIG. 2.

Table 1 below shows a comparison of numerical particle size distribution obtained by microscopical image analysis of brinzolamide suspension samples prepared by high shear homogenization, high pressure homogenization and ball milling:

TABLE 1
Brinzolamide ophthalmic suspension
PARTICLE SIZE DISTRIBUTION BY SPHERICAL DIAMETER

| | Count (%) | | | | | |
|---|---|---|---|---|---|---|
| | High shear | | High pressure | | | |
| size | homogenization | | homogenization | | Ball milling | |
| classes | 1. | 2. | 1. | 2. | 1. | 2. |
| (up to μm) | measurement | measurement | measurement | measurement | measurement | measurement |
| 0.5 | 0.3 | 0.1 | 0.0 | 0.0 | 0.0 | 0.0 |
| 1.0 | 20.3 | 19.5 | 23.0 | 20.6 | 32.7 | 32.0 |
| 1.5 | 16.5 | 16.0 | 20.0 | 17.9 | 22.6 | 22.1 |
| 2.0 | 13.1 | 13.5 | 16.5 | 16.7 | 14.7 | 15.6 |
| 2.5 | 10.5 | 10.9 | 12.2 | 13.1 | 8.8 | 9.3 |
| 3.0 | 7.6 | 8.7 | 9.0 | 10.4 | 5.4 | 5.4 |
| 3.5 | 6.6 | 6.4 | 7.1 | 7.8 | 4.0 | 4.0 |
| 4.0 | 4.8 | 4.7 | 4.5 | 4.6 | 2.7 | 2.5 |
| 4.5 | 4.0 | 4.8 | 3.1 | 3.5 | 2.5 | 2.1 |
| 5.0 | 3.1 | 3.5 | 2.0 | 2.2 | 1.7 | 1.6 |
| 5.5 | 2.6 | 2.7 | 1.1 | 1.4 | 1.1 | 1.3 |
| 6.0 | 2.5 | 1.9 | 0.6 | 0.9 | 1.0 | 0.9 |
| 6.5 | 1.4 | 1.6 | 0.5 | 0.3 | 0.7 | 0.7 |
| 7.0 | 1.4 | 1.2 | 0.2 | 0.3 | 0.5 | 0.6 |
| 7.5 | 1.1 | 1.2 | 0.1 | 0.1 | 0.4 | 0.5 |
| 8.0 | 1.3 | 0.7 | 0.0 | 0.0 | 0.4 | 0.4 |
| 8.5 | 0.9 | 0.9 | 0.0 | 0.0 | 0.4 | 0.3 |
| 9.0 | 1.0 | 0.8 | 0.0 | 0.0 | 0.2 | 0.2 |
| 9.5 | 0.5 | 0.5 | 0.0 | 0.0 | 0.3 | 0.3 |
| 10.0 | 0.6 | 0.5 | 0.0 | 0.0 | 0.1 | 0.2 |
| Mean | 2.60 | 2.58 | 2.01 | 2.11 | 1.87 | 1.87 |
| spherical | | | | | | |
| diameter | | | | | | |
| (μm) | | | | | | |
| No. of | 3957 | 3536 | 4512 | 4680 | 6670 | 7388 |
| particles | | | | | | |
| within | | | | | | |
| range | | | | | | |
| Oversize | 171 | 202 | 0 | 0 | 24 | 34 |
| (No. of | | | | | | |
| particles | | | | | | |
| over 10.0 μm) | | | | | | |

Based on the results presented in FIGS. 1 and 2, and Table 1 above, it can be seen that samples prepared by high shear homogenization and ball milling contain active substance agglomerates larger than 10.0 μm, which is not acceptable for topical ophthalmic preparations. Only the sample prepared by high pressure homogenization in accordance with the present invention is free of agglomerates larger than 10.0 μm.

### Example 1.2

### Wetting Agent—Poloxamer 407

In this example, a brinzolamide ophthalmic suspension was prepared in a 0.05% w/v aqueous solution of Poloxamer 407 as wetting agent by the following methods:
(i) high pressure homogenization (5000 psi/5 cycles using APV high pressure homogenizer)
(ii) high shear homogenization (8000 rpm/30 minutes (Ultra-Turrax high shear mixer)

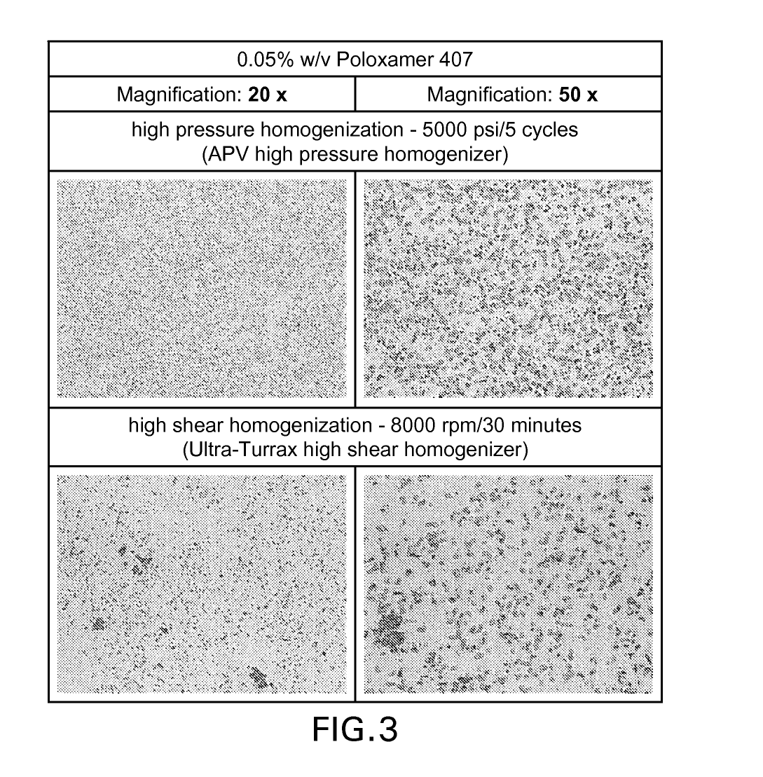

Microscopic images of the suspensions at 20× and 50× magnification are shown in FIG. 3.

### Example 1.3

### Wetting Agent—Tyloxapol

In this example, a brinzolamide ophthalmic suspension was prepared in a 0.05% w/v aqueous solution of Poloxamer 407 as wetting agent by the following methods:
(i) high pressure homogenization (5000 psi/5 cycles using APV high pressure homogenizer)
(ii) high shear homogenization (8000 rpm/120 minutes (Ultra-Turrax high shear mixer)

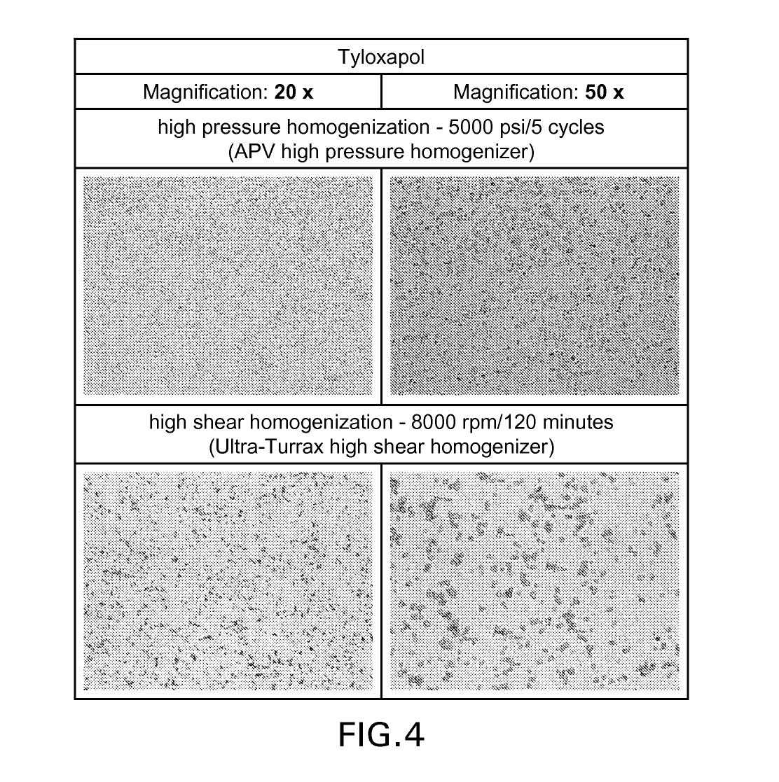

Microscopic images of the suspensions at 20× and 50× magnification are shown in FIG. 4.

From the presented results it can be concluded that high pressure homogenization in accordance with the present invention is the most effective technique for the preparation of agglomerate-free Brinzolamide ophthalmic suspensions.

### Example 2

### Evaluation of Suspension Stability of Brinzolamide Ophthalmic Suspension Prepared by High Pressure Homogenization

A brinzolamide ophthalmic suspension was prepared using Poloxamer 407 by high pressure homogenization at the following processing parameters:

Homogenizing pressure: 5000 psi

No. of cycles: 5 cycles

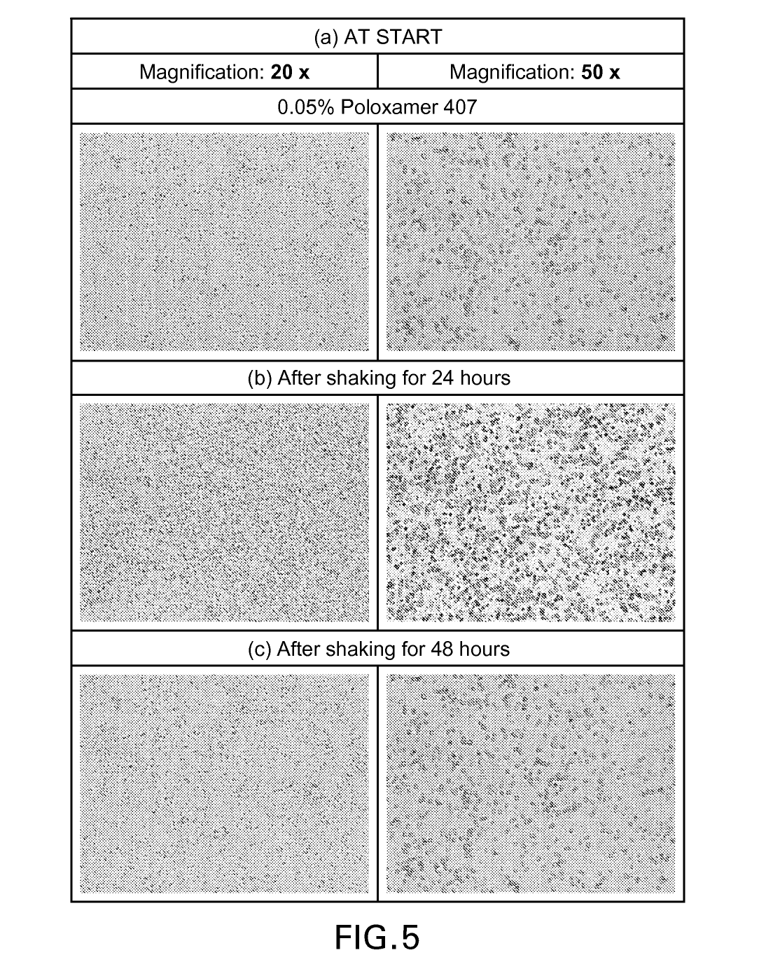

Microscopic images of prepared suspension were taken: (a) at the START point, (b) after shaking on the laboratory shaker (amplitude: 90 min⁻¹) for 24 h, and (c) after shaking on a laboratory shaker (amplitude: 90 min⁻¹) for 48 h. These shaking experiments were performed in order to explore stability of the suspensions (FIG. 5).

Table 2 below shows the numerical particle size distribution obtained by microscopical image analysis of the brinzolamide/Poloxamer 407 ophthalmic suspension sample prepared by high pressure homogenization:

TABLE 2
Brinzolamide ophthalmic suspension
PARTICLE SIZE DISTRIBUTION BY SPHERICAL DIAMETER

| | Count (%) | |
|---|---|---|
| size classes | | After shaking |
| (up to μm) | START | for 48 hours |
| 0.5 | 0.1 | 0 |
| 1.0 | 42.0 | 36.1 |
| 1.5 | 29.1 | 27.9 |
| 2.0 | 17.4 | 18.3 |
| 2.5 | 7.7 | 9.4 |
| 3.0 | 2.5 | 4.9 |
| 3.5 | 0.9 | 2 |
| 4.0 | 0.3 | 0.8 |
| 4.5 | 0.0 | 0.4 |
| 5.0 | 0.0 | 0.1 |
| 5.5 | 0.0 | 0.1 |
| 6.0 | 0.0 | 0.0 |
| 6.5 | 0.0 | 0.0 |
| 7.0 | 0.0 | 0.0 |
| 7.5 | 0.0 | 0.0 |
| 8.0 | 0.0 | 0.0 |
| 8.5 | 0.0 | 0.0 |
| 9.0 | 0.0 | 0.0 |
| 9.5 | 0.0 | 0.0 |
| 10.0 | 0.0 | 0.0 |
| Mean spherical | 1.27 | 1.41 |
| diameter (μm) | | |
| Maximum spherical | 4.76 | 7.32 |
| diameter (μm) | | |
| No. of particles | 8763 | 8623 |
| within range | | |
| Oversize (No. of | 0 | 0 |
| particles over | | |
| 10.0 μm) | | |

Based on the results presented in FIG. 5 and Table 2 above, it can be seen that high pressure homogenization is an effective method of stabilizing brinzolamide suspensions as demonstrated by the shaking experiments.

### Example 3

### Formulation Example

| MATERIAL | % |
|---|---|
| Brinzolamide | 1 |
| Benzalkonium chloride | 0.01 |
| Disodium edetate | 0.01 |
| Sodium chloride | 0.3 |
| Mannitol | 3.3 |
| Poloxamer 407 | 0.05 |
| Carbomer (Carbopol 974P) | 0.45 |
| Sodium hydroxide | 0.2-0.3 |
| Hydrochloric acid | 0.1 |
| Water for injection | Up to 100 |

### Production Steps

a. prepare a slurry containing water for injection, chelating agent (disodium edetate) and viscosity/suspending agent (Carbopol 974P)
b. prepare a solution containing water for injection, tonicity agents (sodium chloride and mannitol) and preservative (benzalkonium chloride)
c. mix the slurry from step (a) and the solution from step (b) and add the pH adjustment agent (sodium hydroxide and/or hydrochloric acid) to bring the slurry to an ophthalmically acceptable pH
d. sterilize the slurry of step (c) by autoclaving
e. prepare a solution containing water for injection and wetting agent (Poloxamer 407) and sterilize by autoclaving
f. suspend sterile brinzolamide in the Poloxamer 407 solution prepared in step (e)
g. subject the solution of step (f) to high pressure homogenization (e.g. microfluidizer or piston-gap homogenizer)
h. mix the slurry prepared in step (d) with the brinzolamide suspension prepared in step (g) under aseptic conditions

In step g, the high pressure homogenization is preferably carried out to effect deagglomeration of aggregates present in the suspension, and does not effect any significant particle size reduction of the drug particles. Hence, the high pressure homogenization is preferably conducted at a pressure of about 100 to about 500 bar, particularly about 150 to about 450 bar, more particularly about 200 to about 400 bar, and preferably about 300 to about 380 bar. Preferably, the high pressure homogenization may be carried out over about 2 to about 10 cycles, preferably about 2 to about 5 cycles, about 3 to about 8 cycles, and more preferably about 4 to about 6 cycles.

In a more preferred embodiment the high pressure homogenization is conducted at pressure of about 200 to about 400 bar, for about 3 to about 7 cycles. Alternatively, the high pressure homogenization is conducted at a pressure of about 300 to about 380 bar for about 4 to about 6 cycles.

1. A process for preparing an ophthalmic formulation for topical application comprising:
(i) an ophthalmic drug,
(ii) at least one wetting agent, and
(iii) an aqueous vehicle
wherein the ophthalmic drug is in aqueous suspension in the formulation, said process comprising:
(a) subjecting a suspension of the ophthalmic drug in an aqueous solution of wetting agent to high pressure homogenization, and
(b) combining the mixture in step (a) with the aqueous vehicle.

2. A process according to claim 1 wherein step (a) comprises microfluidization or piston-gap homogenization.

3. A process according to claim 1 or claim 2 wherein the high pressure homogenization is conducted under pressures and/or number of cycles so as to effect deagglomeration of drug particle aggregates without a significant reduction in particle size of the drug particles.

4-7. (canceled)

8. A process according to claim 1 wherein the suspension of the ophthalmic drug in the aqueous solution of wetting agent is provided by combining a sterile drug with a sterile aqueous solution of wetting agent.

9-10. (canceled)

11. A process according to claim 1 wherein the suspension of the ophthalmic drug in the aqueous solution of wetting agent is sterilized prior to high pressure homogenization.

12. A process according to claim 1 wherein the aqueous vehicle is obtained by:
(i) preparing an aqueous slurry containing an ophthalmically acceptable excipient selected from the group consisting of a chelating agent, preservative, tonicity agent, viscosity/suspending agent, and optionally a buffer, or a mixture thereof,
(ii) adjusting the aqueous slurry to an ophthalmically acceptable pH, and
(iii) sterilizing the slurry.

13. A process according to claim 1 wherein the ophthalmic drug is selected from the group consisting of a prostaglandin, carbonic anhydrase inhibitor, α-adrenergic agonist, non-steroidal anti-inflammatory, anti-fungal agent, antibiotic, corticosteroid, beta-blocker, or a combination thereof.

14. A process according to 1 claim wherein the ophthalmic drug is selected from the group consisting of brimonidine, brinzolamide, dorzolamide, natamycin, ofloxacin, bimatoprost, travoprost, latanoprost, nepafenac, ketoconazole, fluconazole, voriconazole, hydrocortisone, prednisolone, dexamethasone, timolol, levobunolol, betaxolol, or pharmaceutically acceptable salts, and combinations thereof.

15-16. (canceled)

17. A process according to claim 1 wherein the ophthalmic drug has been micronized prior to step (a).

18-19. (canceled)

20. A process according to claim 1 wherein the wetting agent is selected from the group consisting of polyoxypropylene-polyoxyethylene block copolymers (poloxamers), polyethoxylated ethers of castor oils, polyoxyethylenated sorbitan esters (polysorbates), polymers of oxyethylated octyl phenol (Tyloxapol), polyoxyl 40 stearate, fatty acid glycol esters, fatty acid glyceryl esters, sucrose fatty esters, polyoxyethylene fatty esters, and mixtures thereof.

21-33. (canceled)

34. A process according to claim 1 wherein the formulation is substantially free of drug particles having a particle size of over 10 μm.

35. A process according to claim 1 wherein the formulation contains less than 0.02% by weight of drug particles having a particle size of over 10 μm.

36. A process according to claim 1 containing no detectable drug particles having a particle size of over 10 μm.

37. A process according to claim 1 wherein the formulation is substantially free of drug particles having a particle size of 10 μm or more following shaking the formulation at an amplitude of 90/min for 24 hours at room temperature.

38. A process according to claim 1 having less than 0.02% by weight of drug particles having a particle size of 10 μm or more following shaking the formulation at an amplitude of 90/min for 24 hours at room temperature.

39-40. (canceled)

41. An ophthalmic formulation obtainable by the process according to claim 1.

42. An ophthalmic formulation for topical application comprising:
(i) an ophthalmic drug,
(ii) at least one wetting agent, and
(iii) an aqueous vehicle
wherein the ophthalmic drug is in aqueous suspension in the formulation, and the formulation is substantially free of drug particles having a particle size of over 10 μm.

43-50. (canceled)

51. A method of preventing the formation of drug aggregates in an ophthalmic formulation containing an ophthalmic drug suspended in an aqueous vehicle containing at least one wetting agent by the use of high pressure homogenization.

52. The method according to claim 51 wherein the drug is a carbonic anhydrase inhibitor selected from brinzolamide or dorzolamide, or pharmaceutically acceptable salts thereof.
